(19) United States
(12) Patent Application Publication
(10) Pub. No.: US 2013/0035593 A1
(43) Pub. Date: Feb. 7, 2013

(54) GAS ADAPTOR AND METHOD OF USE

(71) Applicants: Fred Lampropoulos, Salt Lake City, UT (US); Graham Taylor, Soddy Daisy, TN (US); Richard Collard, Kaysville, UT (US); F. Mark Ferguson, Salt Lake City, UT (US); Jim Mottola, Salt Lake City, UT (US)

(72) Inventors: Fred Lampropoulos, Salt Lake City, UT (US); Graham Taylor, Soddy Daisy, TN (US); Richard Collard, Kaysville, UT (US); F. Mark Ferguson, Salt Lake City, UT (US); Jim Mottola, Salt Lake City, UT (US)

(73) Assignee: Merit Medical Systems, Inc., South Jordan, UT (US)

(21) Appl. No.: 13/366,019

(22) Filed: Feb. 3, 2012

Related U.S. Application Data
(60) Provisional application No. 61/439,645, filed on Feb. 4, 2011.

(51) Int. Cl. A61B 6/00; A61M 5/31

(52) U.S. Cl. 600/432; 600/431

(57) ABSTRACT

A system for safely delivering gas through a medical device to a patient and a method for using an exemplary system are disclosed. The system may be configured to minimize the risk of delivering an unwanted gas through mix up or mistake. A pneumatic fitting for use in medical procedures is also disclosed. The fitting may be configured to mate exclusively with a specific gas or fluid source.

### CROSS-REFERENCE TO RELATED APPLICATIONS

This application claims priority under 35 U.S.C. §119(e) to U.S. Provisional Application No. 61,439,645 filed on Feb. 4, 2011, titled “CARBON DIOXIDE FITTING AND METHOD OF USE”, the entire contents of which are hereby incorporated by reference.

### TECHNICAL FIELD

The present disclosure relates generally to medical devices. More specifically, the present disclosure relates to a method and apparatus to deliver medical gas to a patient and pneumatic adaptors for a medical gas supply.

### BRIEF DESCRIPTION OF THE DRAWINGS

The embodiments disclosed herein will become more fully apparent from the following description, taken in conjunction with the accompanying drawings. These drawings depict only typical embodiments, which will be described with additional specificity and detail through use of the accompanying drawings in which:

FIG. 1A is a perspective view perspective view of one embodiment of a pneumatic adaptor.

FIG. 1B is a cross sectional perspective view respectively of one embodiment of a pneumatic adaptor.

FIG. 2A is a perspective view of a reservoir device for use in connection with the pneumatic device of FIGS. 1A-1B.

FIG. 2B is a perspective view of a syringe system device for use in connection with the pneumatic device of FIGS. 1A-1B.

FIG. 3A is a plan view of the reservoir device shown in FIG. 2A in connection with the pneumatic device of FIGS. 1A-1B and a gas source.

FIG. 3B is a perspective view of the reservoir device as shown in FIG. 2A in connection with the syringe system device shown in FIG. 2B.

FIG. 4A is a perspective view depicting the step of withdrawing gas from the reservoir device into the syringe system device.

FIG. 4B is a view depicting the step of delivering the gas from the syringe device to a patient.

### DETAILED DESCRIPTION

The phrases “connected to,” “coupled to,” and “in communication with” refer to any form of interaction between two or more entities, including mechanical, electrical, magnetic, electromagnetic, fluid, and thermal interaction. Two components may be coupled to each other even though they are not in direct contact with each other. For example, two components may be coupled to each other through an intermediate component.

The term “fluid” is given its normal definition as a substance that continually flows such as gases and liquids.

Gases, such as carbon dioxide gas, may be used in conjunction with numerous medical procedures. These medical procedures may be for either treatment or diagnostic purposes. The Food and Drug Administration (FDA) has voiced concern about injuries and deaths caused by medical gas mix-ups. Safety features to prevent the wrong gas from being used include gas cylinder markings criteria. Nevertheless, incidents of injury or death due to using the wrong gas in a medical device continue to occur.

Disclosed herein are pneumatic systems which include a pneumatic adaptor for coupling a gas source to a medical device, the medical device being designed to deliver medical gas to a patient. The system may include the medical device as well as a safety feature which only allows the pneumatic adaptor to couple to a medical device for which it was designed. Thus, the pneumatic adaptor may be configured to prevent delivery of the wrong gas to a medical device. Further methods of using the pneumatic system to inject gas into a patient through a medical device are disclosed herein. Additionally, pneumatic adaptors which may couple a medical device to a gas source are also disclosed. The pneumatic adaptor may be configured so that it only couples to a particular gas source and a particular medical device to minimize user error.

Disclosed herein is a pneumatic system which delivers gas from a gas source into a medical device system designed to deliver the gas to a patient. This system may include a safety feature comprising a pneumatic adaptor configured to reduce the risk that the wrong gas will enter the medical device.

Another aspect of the current disclosure are methods of using a pneumatic system to inject gas into a patient for treatment or diagnostic purposes. One embodiment of such a method includes injecting carbon dioxide gas into a patient's body as contrast media.

A further aspect of the current disclosure are pneumatic adaptors that couple a gas source to a medical device designed to deliver gas to a patient. The pneumatic adaptors may be configured to connect a medical device exclusively with a predetermined gas source. The adaptor may be configured to prevent coupling a medical device to a gas source other than the predetermined gas source. Thus, the adaptor may be configured as a safety feature that prevents medical gas mix-ups.

It will be readily understood by one of skill in the art having the benefit of this disclosure that the components of the embodiments as generally described and illustrated in the Figures herein could be arranged and designed in a wide variety of different configurations. Thus, the following more detailed description of various embodiments, as represented in the Figures, is not intended to limit the scope of the disclosure, but is merely representative of various embodiments. While the various aspects of the embodiments are presented in drawings, the drawings are not necessarily drawn to scale.

The disclosure provided herein in connection with any particular figure is analogously applicable to the disclosure provided in connection with other figures. Further, components described and labeled in one figure may be present in the embodiments of other figures whether or not the features are labeled or described in each instance.

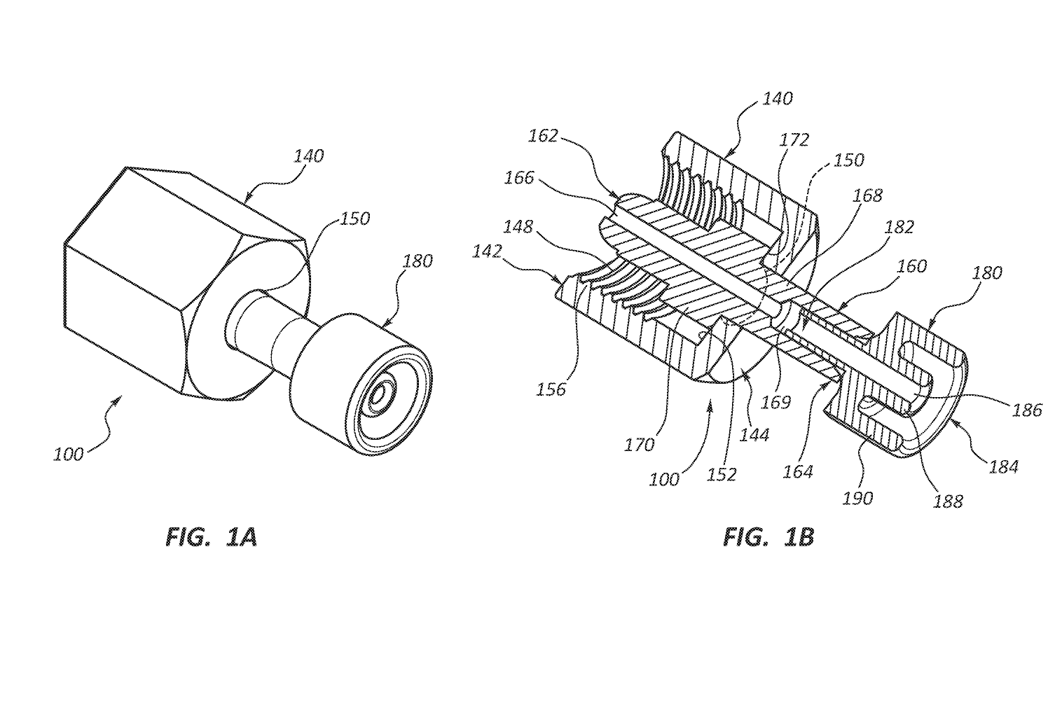

FIG. 1A is a perspective view of one embodiment of a pneumatic adaptor 100 that may couple a regulator, or other source of medical gas, to a medical device. The pneumatic adaptor of the illustrated embodiment includes a nut 140 configured to secure the pneumatic adaptor to a regulator (or other fitting) coupled to a gas source and a connector 180 that may be configured to couple to a medical device or to medical tubing used with a medical device.

FIG. 1B is a cross-sectional perspective view of the pneumatic adaptor of FIG. 1A. In the illustrated embodiment, the pneumatic adaptor 100 comprises three subcomponents: a nut 140, a nipple 160, and a connector 180. The nipple 160 defines a nipple first end 162 and a nipple second end 164. The nipple first end 162 may be configured to mate with a carbon dioxide regulator (not shown). The nut 140 may function to couple the nipple 160 to the regulator. The connector 180 is coupled to the nipple second end 164 of the nipple 160.

The nipple 160 and connector 180 may define a central fluid pathway, essentially running along the center axis of the components. More specifically, the nipple 160 may have a nipple central lumen 166 extending from the nipple first end 162 to the nipple second end 164. The connector 180 may also include a connector central lumen 186 extending from the connector first end 182 to the connector second end 184. The connector central lumen 186 may be in fluid communication with the nipple central lumen 166. Thus, in certain embodiments the connector central lumen 186 and the nipple central lumen 166 may form a fluid pathway between a regulator (coupled to the nipple first end 162) and a medical device coupled to the connector second end 184. It will be appreciated by one of skill in the art having the benefit of this disclosure that, in other embodiments, other components may be present and the nut 140, nipple 160, and connector 180 may be arranged in different spatial configurations.

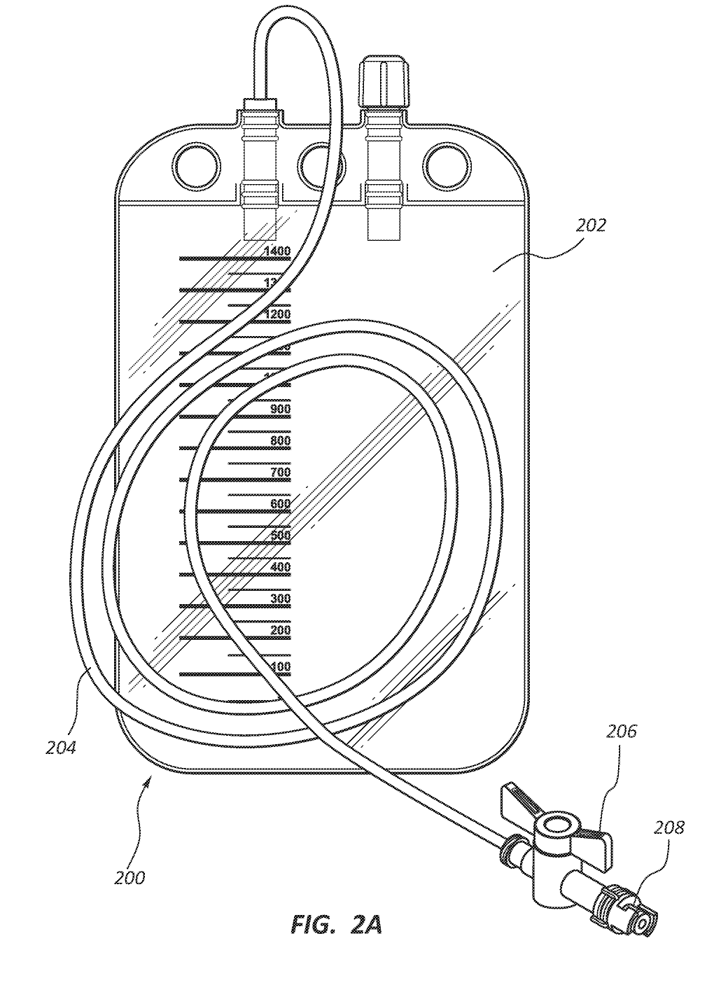

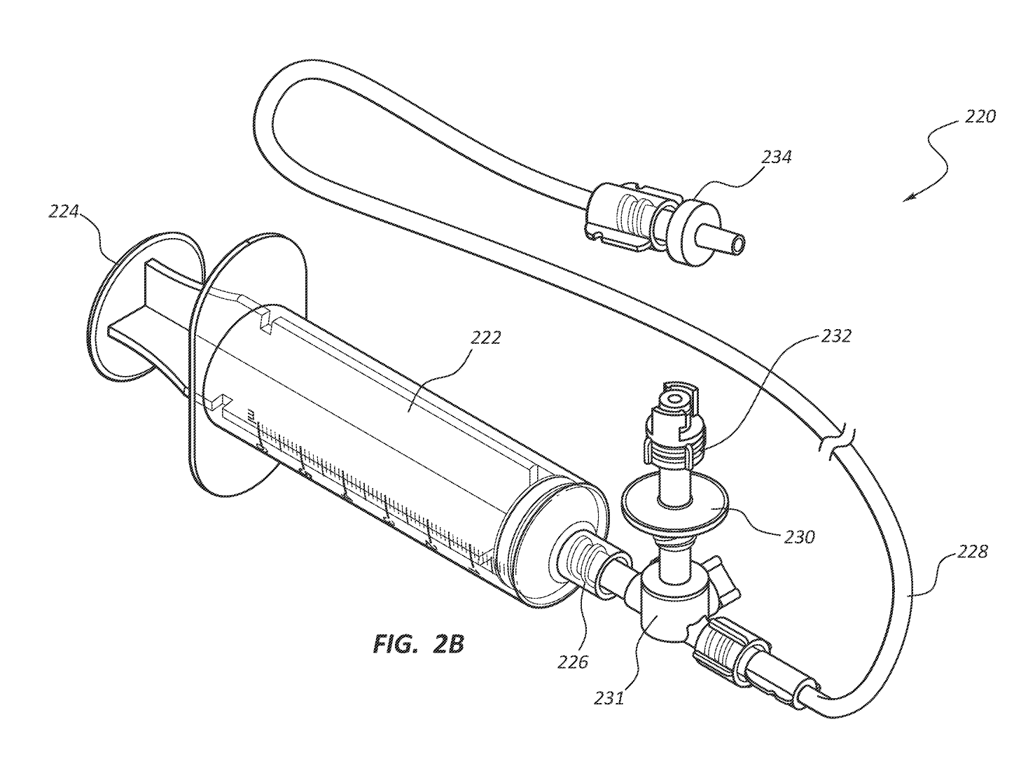

The nipple 160 may be configured to exclusively couple to a regulator, valve, or other fitting that, in turn, is configured for use with a specific type of gas. The connector 180 may be configured to exclusively couple to a gas-specific medical tubing connector. Therefore, the nature of the nipple 160 and connector 180 on the pneumatic adaptor 100 may provide a safety function, as they are configured for the coupling of the proper medical device to the proper gas source. More concisely, a particular embodiment of the pneumatic adaptor 100 may only properly couple to a specific gas source and a specific medical tubing connector that is connected to a medical device designed to use the gas from that gas source. For example, an embodiment of the pneumatic adaptor may be configured to couple a carbon dioxide gas regulator to a carbon dioxide line and reservoir as shown in FIGS. 2-4 which delivers carbon dioxide gas to a patient. The nipple 160 for this embodiment of the pneumatic adaptor may be configured to couple exclusively to a regulator that is configured to couple to a carbon dioxide source. The connector 180 of this embodiment of the pneumatic adaptor may be configured to couple exclusively to the carbon dioxide line and reservoir shown in FIGS. 2-4 or exclusively to medical devices which use carbon dioxide. In contrast, this embodiment of the pneumatic adaptor would not couple to a regulator configured to couple to an oxygen source or a medical device that uses oxygen.

As shown in FIGS. 1A-1B generally, the nut 140 may include a top aperture 150 and a nut bore 148. A portion of the shaft 168 of the nipple 160 may pass through the top aperture 150 and continue through the nut bore 148. The top aperture 150 may have a diameter larger than a shaft 168 of the nipple, thereby allowing the nut 140 to be translatable with respect to the shaft 168 of the nipple in the axial direction. Consequently, the nipple first end 162 may emerge through top aperture 150 of the nut 140. Furthermore, the top aperture 150 may have a smaller diameter than a flange 170 located on a portion of the nipple 160. The fitting 100 can therefore be configured such that the nut 140 may restrain the axial movement of the nipple 160 due to the interaction between the nut 140 and the flange 170.

The connector 170 may be coupled to the nipple second end 164. In some embodiments the nipple 160 will have a bore within the nipple second end 164, coaxially aligned with the nipple central lumen 166 extending from the nipple second end 164. The bore within the nipple second end 164 may have a diameter configured to receive the connector first end 182. In some embodiments the connector first end 182 may be rigidly attached to the nipple 160, such as by bonding or an interference fit between the connector 180 and a bore within the nipple second end 164. In still other embodiments, the connector 180 may be coupled to the nipple 160 by the interaction of mating grooves and ridges on the components which allow the components to rotate with respect to each other (around the axis of the fitting) but which restrain the axial movement of the two components with respect to each other. Further, the connector 180 may be coupled to the nipple 160 such that some axial displacement is allowed.

In the embodiment shown in FIGS. 1A-1B, the connector first end 182 is rigidly coupled to the bore of the nipple 160 at the nipple second end 164. However, in other embodiments the connector 180 may be coupled to the nipple 160 in a non-fixed manner, such that the components have some degree of freedom relative to each other. For instance the two components may be coupled in such a manner as to allow some axial translation or couple such that the two components may rotate relative to each other. Also, in the illustrated embodiment, the nut 140 is not fixed to any other component and is therefore allowed to translate along the shaft 168 of the nipple 160 between the flange 170 and the larger portion of the connector 180. In other embodiments, however, the nut 140 may be rigidly fixed to the nipple 160, or only allowed one degree of freedom, such as rotation or translation.

The nut 140 may include internal threads 156 configured to mate with male threads on a medical gas regulator. The nut 140 may therefore be coupled with a regulator connection location, such that the nut 140 secures the nipple first end 162 to the regulator connection. Axial movement of the nipple 160 may be constrained by contact with the regulator connection at the nipple first end 162 and contact between the nut 140 and the flange 170.

The nut 140 may include a nut bore 148 defining an inside diameter of the nut 140. The shaft 168 of the nipple may pass through the nut bore 148. Further the nut 140 may include threads. The threads may be internal threads 156, as illustrated in FIG. 1B, along a portion of the inside diameter of the nut bore 148. The internal threads 156 may be configured to mate with external threads on a gas regulator. In one embodiment the internal threads 156 may be sized such that they mate exclusively with the threads of a carbon dioxide regulator. Twisting the nut 140 onto the external threads of a regulator may couple the nut 140 to the regulator and prevent movement of the nut 140 with respect to the regulator (with the exception of twisting the nut 140 along the threads of the regulator).

The nut bore 148 of the nut 140 may define an upper surface 152. The upper surface 152 may configured to interact with a flange 170 of a nipple 160 when the two components are used in conjunction with each other. The nut 140 may exert an axial force on the flange 170 of the nipple 160 when the nut 140 is tightened onto the threads of a regulator, the axial force acting to compress the nipple first end 162 against a mating fitting of the regulator.

The internal threads 156 of the nut 140 may be designed, in some embodiments, to couple only to a particular type of gas regulator. Gas regulators are designed with specific threads (i.e.,a size and type of thread is specific to a certain gas). Thus, the internal threads 156 of the nut 140 may be sized such that the nut 140 is configured to couple only to a regulator for a particular gas. The internal threads 156 may extend along a portion of the inside diameter of the nut bore 148, in other words, the internal threads 156 may or may not extend along the entire axial length of the nut bore 148. Further, in some embodiments the threads will be configured to mate with fittings unique to carbon dioxide delivery while in other embodiments the threads will be those used for other fluids, such as oxygen, nitrogen, helium, medical air, a mixture of these gases, or any other fluid.

As previously indicated, the nut 140 may include a top aperture 150. In some embodiments the top aperture 150 extends from the upper surface 152 of the nut bore 148 to the nut second end 144.

The portion of the nipple 160 near the nipple first end 162 may be configured with multiple diameters and shapes. In some embodiments the shape of this portion of the nipple 160 is configured to mate with another gas fitting. For example, one carbon dioxide regulator standard is configured to receive a nipple with two specific outside diameters at different points along the axial direction of the nipple. Thus, the size and features of the nipple may be configured to mate with any number of gas fitting as defined by engineering standards or practice. It will also be appreciated that in some embodiments these standards define both the shape of the nipple 160 and the threads used on the corresponding nut 140.

The nipple 160 may include a flange 170. The flange 170 may define a shoulder 172 which may be configured to interact with the upper surface 152 of the nut bore 148 of a nut 140. An axial force acting on the shoulder 172 of the nipple 160 may force the nipple first end 162 into contact with another pneumatic fitting. Further, such an axial force may compress the nipple 160 between the shoulder 172 and the nipple first end 162 such that the nipple first end 162 is partially deformed by contact with the mating fitting, thereby forming a seal.

The nipple 160 may further be configured with a nipple bore 169 in the nipple second end 164. As described above, this bore may be sized to accommodate a connector 180.

Referring again to the connector shown in FIGS. 1A and 1B, it will be appreciated that the connector 180 may be any pneumatic or other connector known in the art. Further, the connector may be coupled to the nipple 160 in any manner known in the art. In some embodiments the connector 180 will be bonded to the nipple 160 in such a manner as to minimize gas leakage at the connection.

In some embodiments the connector 180 may be a “quick connector” type coupling. For example, the connector 180 may have a connection portion 188 configured to mate with another fitting. This portion may be a “male” type fitting or a “female” type fitting. In some embodiments one or more o-rings may be used in conjunction with the fitting to create a seal. The connector 180 may also include a connector collar 190 which may be allowed to rotate independently of other portions of the connector 180. The collar portion may include cut out portions or barbs designed to interact with components on a mating fitting to secure the two fitting together.

In one exemplary embodiment of a connector the connection portion 188 is configured to slide onto (either within or around) a mating connection portion of another fitting. The seal between these two components may be enhanced by one or more o-rings. The connector collar 190 of the exemplary embodiment contains cutaway portions with barbs which interact with the mating fitting. When the connector collar 190 is rotated, the barbs come into contact with mating barbs and secure the connector 180 to the mating fitting. It will be appreciated that the connector collar 190 need not be allowed to rotate in all embodiments. Further in certain embodiments the connector collar 190 on the fitting may rotate but the analogous collar portion of the mating fitting may not; the mating fitting may have a rotating portion while the connector collar 190 does not rotate; both the fitting and the mating fitting may have rotating portions; or neither the fitting nor the mating fitting have rotating portions.

The type of connector 180 used may be designed such that in one embodiment it only mates with certain types of medical equipment. In such embodiments the risk of coupling the connector 180 to the wrong device may be minimized. For example, certain types or sizes of connector may be utilized in medical procedures which employ carbon dioxide while other types or sizes are used for oxygen. In some embodiments the size and type of connector will be selected based on the intended use of the medical equipment to which the connector will be coupled. That is, medical equipment intended for use with carbon dioxide will be coupled to a connector sized for carbon dioxide lines and fittings. This system may reduce the risk a practitioner will mistakenly couple a device intended for use with one gas to a supply of a different gas.

FIG. 2A shows a reservoir device 200 for use in connection with the pneumatic adaptor 100 described above. The illustrated device comprises a reservoir 200 configured to receive and store gas for use in medical procedures. The reservoir 200 comprises a gas reservoir 202, a first section of medical tubing 204, a valve, such as a stopcock 206, and a gas reservoir connector 208. While depicted in this embodiment as a bag, the gas reservoir 202 may be another type of gas-tight container. The gas reservoir connector 208 may be configured to mate with a connector 180 of the pneumatic adaptor 100. The gas reservoir connector 208 may be configured such that it may couple only to a pneumatic adaptor 100 designed to couple to a regulator for the proper gas source for a specific medical device. Thus, if the proper pneumatic adaptor 100 is used, the medical device may only be filled with gas from the correct gas source.

The gas reservoir can then be used in connection with medical devices such as the syringe device 220 of FIG. 2B. In the illustrated embodiment, the syringe device 220 includes a syringe plunger 224 within a syringe barrel 222. A syringe nozzle 226 is at the end of the syringe barrel 222 and is coupled to a second section of medical tubing 228. Both the syringe nozzle 226 and the second section of medical tubing 228 are coupled to a first one-way valve 230, which is, in turn, coupled to a syringe device connector 232. The first one-way valve 230 may regulate flow into and out of the syringe barrel 222. For example, in certain embodiments, the first one-way valve 230 allows gas entering the syringe barrel 222 to enter exclusively through the syringe device connector 232 and gas leaving the syringe to flow exclusively through the second section of medical tubing 228. The illustrated embodiment further includes a second valve 231 which also selectively allows gas entering the syringe barrel 222 to enter exclusively through the syringe device connector 232 and gas leaving the syringe to flow exclusively through the second section of medical tubing 228. The second valve 231 also prevents gas from exiting syringe device 220 through the second section of medical tubing 228 while the syring barrel 222 is being filled with gas. The second valve 231 may be configured to selectively toggle between two or more fluid paths. In other embodiments, the syringe device 220 may be configured with only the first one way valve 230, only the second valve 231, or some other mechanism. A medical device may be coupled to the second section of medical tubing 228 (represented here by component 234).

In some embodiments the syringe device connector 232 may be configured to mate with the gas reservoir connector 208 of the gas reservoir 200. (It is noted that this may mean that syringe device connector 232 is identical to connector 180. For example, if connector 180 were a male fitting configured to be used with female gas reservoir connector 208, in order for syringe device connector 232 to also connect with gas reservoir connector 208, syringe device connector 232 may be a male fitting identical to fitting 180. It will be appreciated in such embodiments fitting 180 could not mate directly to syringe device connector 232.) Further, syringe device connector 232 may be designed with an integral valve component (not pictured). In those embodiments, a valve may be located within syringe device connector 232, the valve configured to be open when the fitting is coupled to another fitting and closed when the fitting is uncoupled. Furthermore, in some embodiments the combination of fittings and medical devices reduces the risk the device will be used improperly as some embodiments prevent the user from connecting the medical devices and medical tubing in an improper configuration.

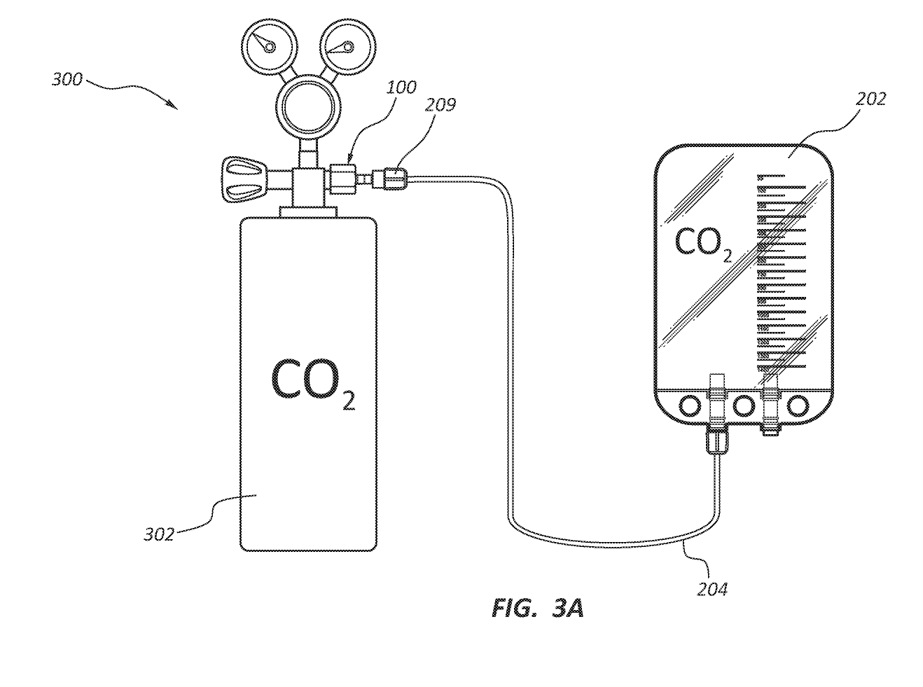

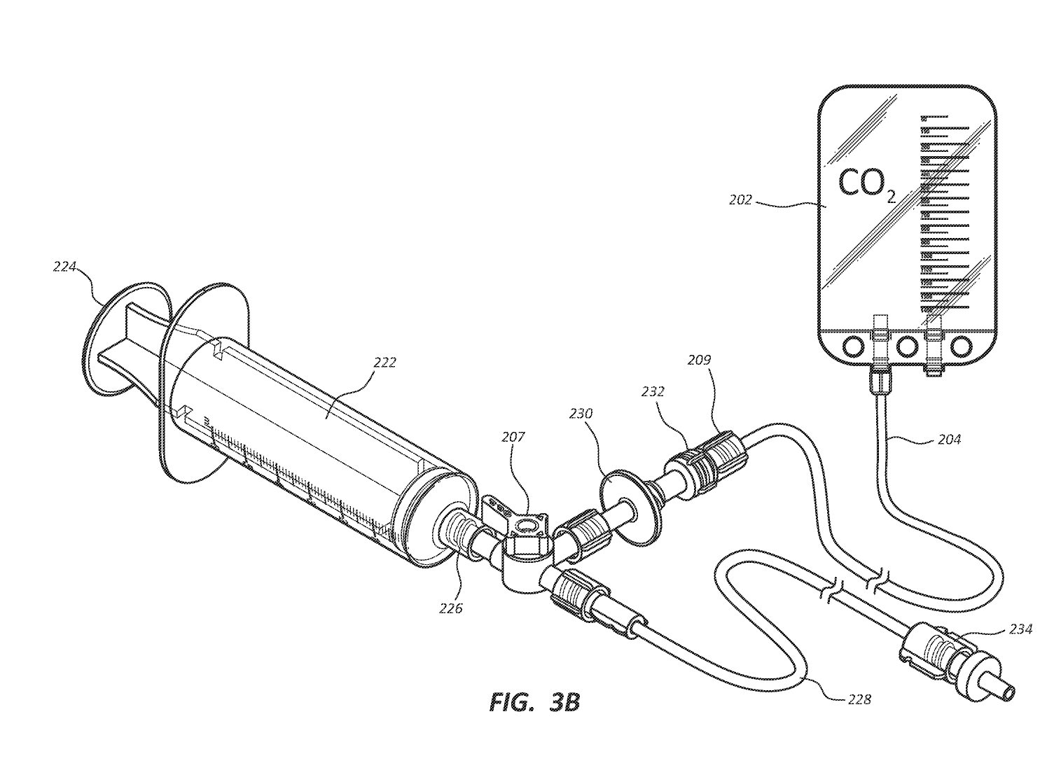

FIG. 3A depicts one embodiment in which the gas reservoir device 200 may be connected to the pneumatic adaptor 100 which in turn is connected to a carbon dioxide regulator. This may be done by connecting the connector 180 of the pneumatic adaptor 100 to a gas reservoir connector 209 (which may be an alternative embodiment of gas reservoir connector 208 as shown in FIG. 2A) of the gas reservoir device 200. When the practitioner opens the regulator, gas will flow through the first section of medical tubing 204 into the gas reservoir 202. It some embodiments, a stopcock, such as stopcock 206 of FIG. 2A may also be positioned on the medical tubing 204. Once the regulator and any other valves are open, pneumatic adaptor is placed in fluid communication with the gas reservoir. When the gas reservoir 202 contains a sufficient amount of gas, the regulator and/or stopcock may each be closed. The connectors 180 and 208 may then be separated.

Furthermore, in some embodiments one or more indicators may be positioned along the fluid path, such as along medical tubing 204 or 228. Indicators may be configured to detect the presence or absence of a particular gas or fluid. For example, an indicator may comprise a portion configured to change color in the presence of oxygen. In the event oxygen or medical air were passed through or occupied the fluid path, the indicator would detect the oxygen and change color. Such an indicator may be configured to provide visual indication of a gas mix-up, such as if oxygen were fed into a line configured for carbon dioxide transmission. The indicator may also be configured to revert back to its present color if the undesired gas were replaced by carbon dioxide, indicating the gas mix-up had been corrected and the unwanted gas had been purged or removed from the fluid path. In some embodiments the indicator may be integral with the connector 100. Additionally, in some embodiments the connector 100 may comprise a pressure relief mechanism, such as a valve configured to release gas if the pressure supplied to the connector 100 from the source is higher than that for which the system (e.g. components such as the gas reservoir 202) is configured for use.

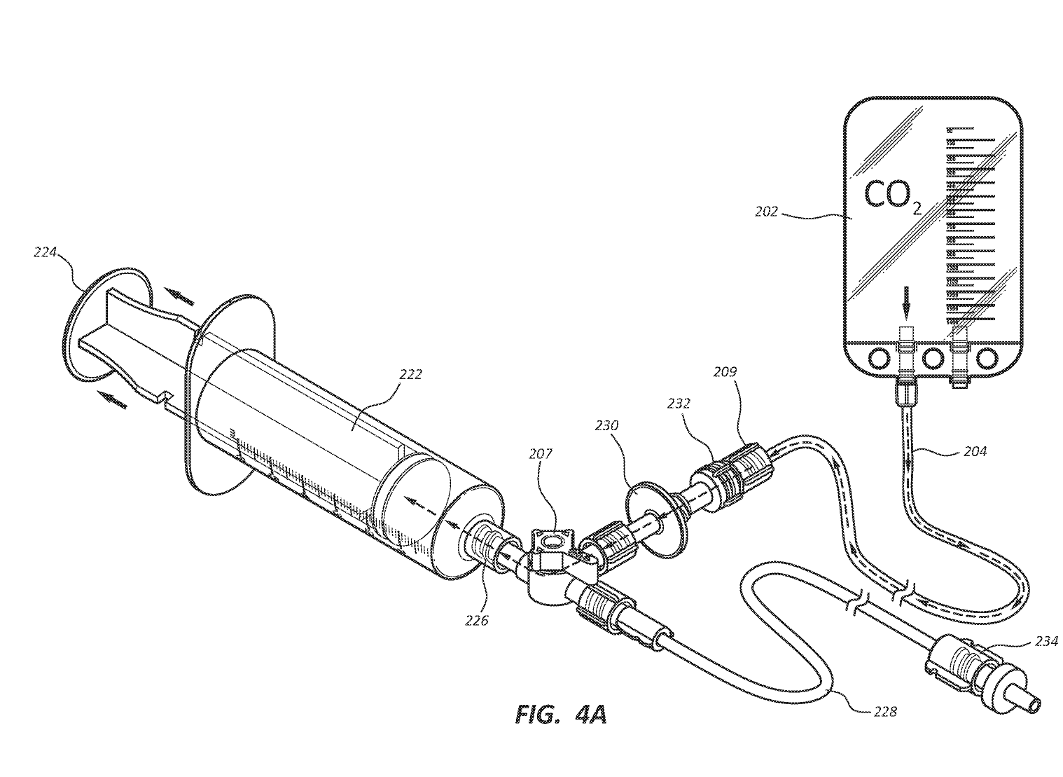

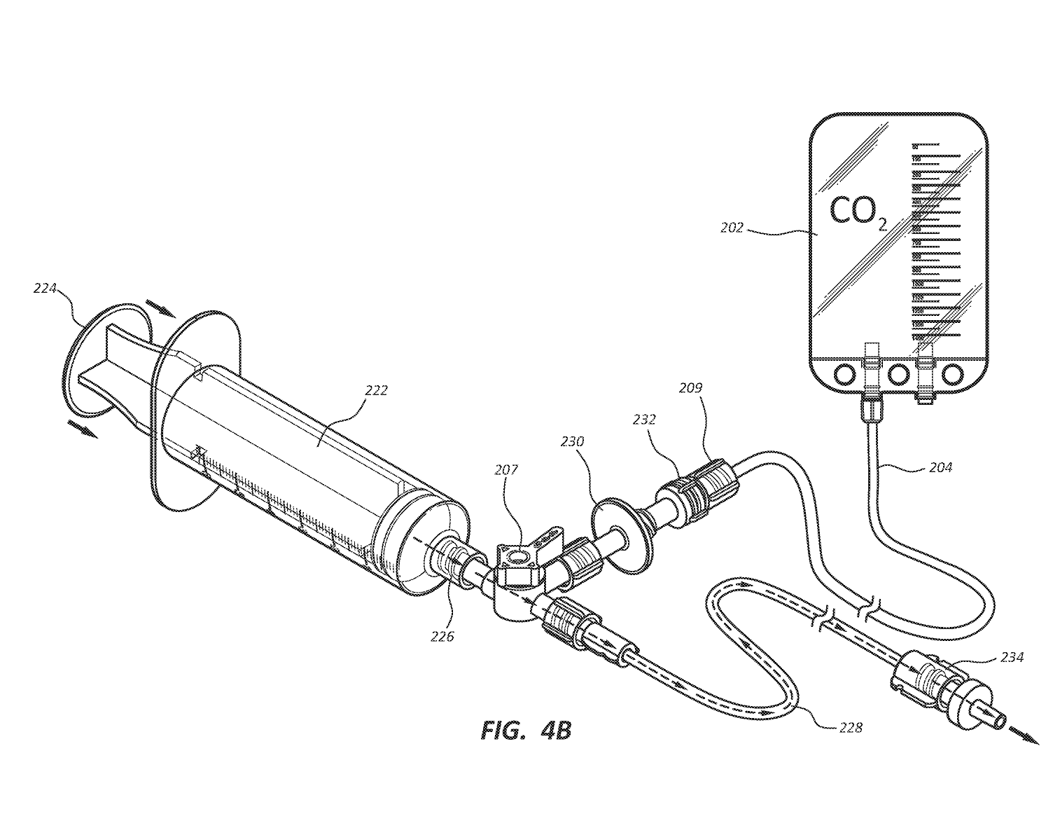

Having filled the gas reservoir 202, the practitioner may uncouple the gas reservoir connector 208 from the pneumatic adaptor 100 in order to fill the syringe device 220 with gas. To do this, the practitioner may couple the gas reservoir connector 208 of the gas reservoir device 200 to the syringe device connector 232 of the syringe device 220 as shown in FIG. 3B. The practitioner may also couple a second section of medical tubing 228 to the syringe nozzle 226 on one end and to the component 234 which can be coupled to the patient on the other end. The practitioner may then draw back the syringe plunger 224 of the syringe device 220 as illustrated in FIG. 4A and fill the syringe barrel 222 with gas from the gas reservoir 202. In the exemplary embodiment the first one-way valve 230 is configured to allow gas to enter the syringe only through the syringe device connector 232.

Once a sufficient amount of gas is in the syringe barrel 222, the practitioner may apply positive pressure downward on the syringe plunger 224 as depicted in FIG. 4B, forcing the gas from the syringe barrel 222 into the second section of medical tubing 228. The valve 230 may be configured to force all gas exiting the through syringe nozzle 226 into the second section of medical tubing 228. The gas may then travel through the second section of medical tubing 228 into component 234, which may be coupled to a patient.

In some instances, a gas may be supplied from a tank or other source through a regulator as depicted in FIG. 3A. In other instances, a gas may be supplied through tubing or other connector that links the gas source, such as a tank, to point of access, such as a valve some distance from the gas source.

Component 234 may connect to any other medical device that is coupled to a patient. (Note: Component 234 represent a connector, not a medical device configured to be coupled directly to a patient.) In one exemplary embodiment, a physician may use the system disclosed herein to deliver carbon dioxide to the body for use as contrast media. Use of the fittings and systems disclosed herein in connection with such a procedure may prevent the accidental use of a non-compatible fluid during the procedure.

FIGS. 4A-4B illustrate the steps of an exemplary method of using the syringe device 220 in which the gas is transferred from the gas reservoir device 200 to the syringe device 220 for delivery to a patient. In the exemplary method illustrated in FIG. 4A, a stopcock 207, coupled to the first section of medical tubing 204 between the gas reservoir 202 and the syringe device connector 232, is configured to exclusively allow gas to enter the syringe barrel 222 from the first section of medical tubing 204 when the stopcock 207 is in a first position. In this example, the stopcock 207 may therefore be placed in a first position which puts the first section of medical tubing in fluid connection with the syringe device 220. When the syringe plunger 224 is actuated, negative pressure is created within the syringe barrel 222 which draws the gas from the gas reservoir 202, through the first section of medical tubing 228, and into the syringe barrel 222. At this point, the syringe device 220 is loaded and ready to deliver the gas to a patient as depicted in FIG. 4B.

FIG. 4B illustrates an exemplary method for using a syringe device, as illustrated in FIG. 2B to delivering carbon dioxide to a patient. To ready the syringe device to deliver carbon dioxide to a patient, the stopcock 207 may next be moved to the second position which blocks the fluid connection between the first section of medical tubing and the syringe device. Therefore, gas may not be forced back into the gas reservoir 202, instead of toward the patient, when positive pressure is applied to the syringe plunger 224 (i.e. the syringe plunger 224 is depressed). When the syringe plunger 224 is pushed into its original position, gas is forced out of the syringe barrel 222, through the second section of medical tubing 228, and into the patient's vasculature.

In an alternative exemplary method, the stopcock 207 is not turned because valve 230 is a one-way valve which prevents carbon dioxide from returning back into the first section of medical tubing 204 after passing into the syringe barrel 222.

It will be appreciated that the disclosure of the pneumatic system and its method of use applies to various types of connectors for use with various fluids, for example, carbon dioxide, oxygen, nitrogen, helium, medical air, nitrous oxide, or mixtures thereof. It will also be appreciated that the gas may be delivered to body parts and body cavities such as lungs, the gastrointestinal tract, or the abdominal cavity.

While specific embodiments of pneumatic fittings and systems for use in connection with those fittings have been illustrated and described, it is to be understood that the disclosure provided is not limited to the precise configuration and components disclosed. Various modifications, changes, and variations apparent to those of skill in the art may be made in the arrangement, operation, and details of the methods and systems disclosed, with the aid of the present disclosure.

Without further elaboration, it is believed that one skilled in the art can use the preceding description to utilize the present disclosure to its fullest extent. The examples and embodiments disclosed herein are to be construed as merely illustrative and exemplary and not a limitation of the scope of the present disclosure in any way. It will be apparent to those having skill in the art that changes may be made to the details of the above-described embodiments without departing from the underlying principles of the disclosure herein.

We claim:

1. A pneumatic system, comprising:
a pneumatic adaptor, comprising:
a nipple comprising a shaft including a nipple first end and a nipple second end, the nipple first end configured to mate with a gas source;
a connector including a connector first end and a connector second end, the connector first end coupled to the nipple second end and the connector second end configured to mate exclusively with a gas-specific medical tubing connector; and
a nut configured to couple the nipple to the gas source comprising:
a bore through which the shaft of the nipple passes; and
an aperture through which the first end of the nipple emerges; and
a first section of medical tubing comprising a first gas-specific medical tubing connector coupled to the second end of the connector on the pneumatic adaptor.

2. The pneumatic system of claim 1, further comprising a gas reservoir wherein the first section of medical tubing is coupled to the gas reservoir permitting fluid communication between the pneumatic adaptor and the gas reservoir.

3. The pneumatic system of claim 1, further comprising a syringe including a syringe connector, wherein the first medical tubing connector is configured to couple to the syringe connector.

4. The pneumatic system of claim 3, further comprising a second section of medical tubing coupled to a syringe nozzle on the syringe providing for fluid communication between the syringe and a patient.

5. The pneumatic system of claim 4, further comprising a stopcock positioned between the syringe and the first section of medical tubing, the stopcock being configured such that when the stopcock is in a first position, gas from the gas reservoir exclusively enters the syringe through the first section of medical tubing and when the stopcock is in a second position, gas in the syringe exits the syringe through the second section of medical tubing.

6. The pneumatic system of claim 1, further comprising an indicator configured to detect the presence or absence of a particular gas or fluid.

7. A method for injecting gas into a patient, comprising:
coupling a pneumatic adaptor to a specific gas source, the pneumatic adaptor comprising a first end configured to mate exclusively with the specific gas source;
coupling a first section of medical tubing to a second end of the pneumatic adaptor, the second end comprising a connector specific to the gas from the gas source, and the first section of medical tubing being coupled to a gas reservoir;
accessing the gas source such that gas flows through the pneumatic adaptor into the gas reservoir via the first section of medical tubing;
uncoupling the first medical tubing connector from the pneumatic adaptor;
coupling the first section of medical tubing to a syringe connector coupled to a syringe, the syringe comprising a syringe plunger, syringe barrel, and syringe nozzle;
coupling a second section of medical tubing to the syringe nozzle and to the patient;
actuating the syringe plunger to draw gas from the gas reservoir and into the syringe barrel; and
applying positive pressure to the syringe plunger until gas is forced into the patient.

8. The method of claim 7, wherein a nut is coupled to the gas source using internal threads to secure the pneumatic adaptor onto the gas source.

9. The method of claim 7, wherein the gas is selected from at least one of the following: carbon dioxide, helium, medical air, nitrogen, nitrous oxide, and oxygen.

10. The method of claim 7, further comprising confirming the presence or absence of the specific gas by observing an indicator configured to detect the presence or absence of the specific gas.

11. A carbon dioxide pneumatic adaptor, comprising:
a nipple, comprising:
a shaft comprising a nipple first end and a nipple second end, the nipple first end configured to mate exclusively with a carbon dioxide gas source;
a nipple central lumen running from the nipple first end to the nipple second end;
a flange positioned on the shaft between the nipple first end and the nipple second end;
a connector, comprising:
a connector first end coupled to the nipple second end;
a connector second end configured to mate exclusively with a gas-specific medical tubing connector;
a connector central lumen running from the connector first end to the connector second end such that the nipple and connector are in fluid communication; and
a nut coupled to the nipple, the nut configured to couple the nipple to a carbon dioxide gas source and the nut limits axial movement of the nipple by abutment with the flange positioned on the shaft of the nipple.

12. The carbon dioxide adaptor of claim 11, wherein the nut comprises internal threads that are configured to mate with a carbon dioxide regulator.

13. The carbon dioxide adaptor of claim 11, wherein the connector lumen and the nipple lumen are configured to form a fluid pathway between the carbon dioxide gas source and a section of medical tubing coupled to the connector second end.

14. The carbon dioxide adaptor of claim 11, wherein the connector first end is rigidly attached to the nipple by bonding the connector first end to the nipple second end.

15. The carbon dioxide adaptor of claim 11, wherein the connector first end is rigidly attached to the nipple by an interference fit between the connector first end and the nipple second end.

16. The carbon dioxide adaptor of claim 11, wherein the connector first end is rigidly attached to the nipple through the interaction of mating grooves and ridges on the connector first end and the nipple.

17. The carbon dioxide adaptor of claim 11, wherein the connector is coupled to the nipple such that some axial displacement is allowed.

18. The carbon dioxide adaptor of claim 11, further comprising further comprising an indicator configured to detect the presence or absence of carbon dioxide.
